(19) United States
(12) Patent Application Publication
(10) Pub. No.: US 2003/0181394 A1
(43) Pub. Date: Sep. 25, 2003

(54) Pharmaceutical composition

(76) Inventors: Ian Stuart Pardoe, Birmingham (GB); Christopher Edward Hartley, Birmingham (GB)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/344,812

(22) PCT Filed: Mar. 5, 2001
(86) PCT No.: PCT/GB01/00946

(30) Foreign Application Priority Data
Mar. 6, 2000 (GB) 0005382.7

(51) Int. Cl. A61K 31/70

(52) U.S. Cl. 514/25

(57) ABSTRACT

A pharmaceutical composition comprising floridoside for use in the therapeutic or prophylactic treatment of neoplastic disease and a range of viral infections, particularly viruses of the Herpes family.

The invention relates to a pharmaceutical composition useful in the therapeutic or prophylatic treatment of a range of viral conditions or neoplastic disease.

A paper by Meng, Rosell and Srivastava, 1986, Carbohydrate Research, 161:171-180 discloses carbohydrate compounds called floridosides, and isomeric forms of the compound called isofloridosides. Floridoside (α-D-galactopyranosyl-(1→2) glycerol) and isofloridoside are complex carbohydrate compounds. Isofloridoside exists in two isomeric forms, Disofloridoside (α-D-galactopyranosyl-(1→1)-D-glycerol) and L-isofloridoside (α-D-galactopyranosyl-(1→1)-L-glycerol). These compounds can be separated by conventional gas chromatography and mass spectrometry methods. The invention is based on the discovery that such compounds have antiviral activity.

We have shown that a water soluble extract from the marine red algae palmaria palmata is effective for inhibiting viral activity. As floridosides and their derivatives comprise 2-20% by weight of palmaria palmata it has been postulated that floridosides represent the active component of the marine algae.

Our investigations also suggest that the floridoside is also effective against neoplastic diseases, i.e. cancers.

In one aspect the invention provides the use of floridoside in the treatment of viral infections and neoplastic disease.

In a second aspect the invention there is provided a pharmaceutical composition comprising a floridoside for use in treating treatment of viral infections and neoplastic disease.

By the term floridoside we mean a floridoside itself and its water soluble or water miscible derivatives such as salts, esters and the like; and the isomeric forms thereof.

The composition may be formulated for application in any suitable way, e.g. as a capsule, a tablet, suppository, injectable solution, topical cream; or the like. Preferably the composition is adapted for administration by oral ingestion.

The pharmaceutical composition is particularly suitable for administration in the treatment of viruses of the Herpes family.

In a further aspect of the invention there is provided a method of preparing floridoside by extraction from palmaria palmata, the method comprising drying the palmaria palmata to a predetermined water content, comminuting the material to a fine powder and dissolving the powder in water.

If necessary, the method may include the step of storing the powder at or below room temperature. Preferably, the method also includes the step of centrifuging the solution and recovering the supernatant.

The dried material is preferably mixed with water in the ratio of 50 g:300 ml.

In yet another aspect the invention provides a method of treating viral infections or neoplastic disease in human or animal subjects, comprising applying to the subject a pharmaceutical preparation of a floridoside. Preferably the application is ingestion.

The rate of application may be determined by routine experimentation.

According to this invention floridosides and isofloridosides are effective in the treatment of viruses of the herpes family including herpes simplex type 1, herpes simplex type 2, varicella zoster, cytomegalovirus, human herpes type 6 and human herpes type 8. The anti-viral activity of these water soluble preparations may also be effective against other viruses.

The Herpes Simplex Virus

The herpes simplex virus (HSV) is a large (150-200 nm) DNA virus which consists of approximately 152,000 base pairs of double stranded DNA encapsulated in a proteinaceous capsid. The capsid is surrounded by a less well defined structure known as the tegument. The virus is contained in a host cell derived lipid bilayer which is studded with virus specific glycoproteins and integral membrane proteins.

Genital Herpes

Recurrent herpes genitalis is estimated to affect over 45 million people in the USA alone. (Fleming et al NEJM Oct 97). The incidence is increasing, leading to a massive reservoir for transmission of infection to sexual partners and new-born infants. Once infected, over 95% of patients who have proven primary herpes genitalis have had at least one recurrence with an average of 5-6 recurrences per year. Approximately 25% of people will have recurrences at monthly intervals accompanied by extremely troublesome physical discomfort and (often) psychological upset. Current therapy for genital herpes revolves around psychological support, education, barrier methods of contraception and treatment on a regular basis with the expensive anti-viral drugs such as Aciclovir, Penciclovir and Famciclovir. These are not tolerated by all sufferers and in some cases are ineffective.

Genital Herpes in Pregnancy

Herpes simplex infection may be spread from mother to foetus. This usually occurs during parturition but can rarely occur in-utero. The rate of transmission depends on whether the mother is suffering primary herpes, where the transmission rate is 50% or recurrent herpes where the transmission rate is 8%. If any lesions are visible during the onset of labour, this would be an indication for undertaking a caesarean section.

Neonatal Infection

50-60% of infants with neonatal Herpes simplex virus infection are born to mothers with no history of genital herpes infection. Neonatal herpes simplex virus infection is very serious. Over 70% of cases present infections localised to the eyes, skin or mouth within 3-30 days of birth. Unfortunately, over three quarters of those infected will progress to disseminated or CNS involvement which carries a very high morbidity and mortality.

Herpes Encephalitis

Acute encephalitis caused by herpes simplex virus is a common and often fatal infection. The herpes viruses have a role in other pathology.

(i). Neurological Diseases: Bells Palsy

There is now compelling evidence to suggest that Bells palsy is caused by an acute herpes simplex infection of the facial nerve. In a study by Murakami (Annals of Internal Medicine 124 pt 1 27-30, January 1996) PCR of endoneural fluid revealed HSV genomes in 11/14 patients while in healthy controls and those with Ramsay Hunt syndrome the virus could not be detected. A recently published placebo controlled double blind trial treating those with Bells palsy with either prednisolone alone or prednisolone with acyclovir showed a very statistically superior outcome when using acyclovir leading the researchers to suggest that bells palsy is probably caused by herpes simplex. (Adour et al Annals of Otology Rhinology and Laryngology 105 (5) 371—May 8, 1996).

(ii). Senile Dementia and Organic Brain Disease

As early as 1975 a link between herpes simplex and senile dementia was suggested. (Libikova Acta Virologica 19(6): 493-5, 1975). In this study, 47% of patients suffering with senile dementia had neutralising antibodies to HSV 1 in their cerebrospinal fluid, compared to none of mentally retarded adolescents. Serum levels of antibodies were elevated in 61% of demented patients compared with 31% of normal controls. More recent work (Jamieson et al Journal of Pathology 167(4): 365—Aug. 8, 1991) used PCR (polymerase chain reaction) to detect thymidine kinase from viral DNA. There was a significant anatomically specific presence of herpes simplex in the brains of those with senile dementia with virus being commonly detected in the hippocampus and the cortex but universally absent in the occipital cortex.

(iii). Inflammatory Bowel Disease

An interesting study by Wakefield (Journal of Medical Virology 1992 Vol. 38 3: 183-190) demonstrated herpes virus DNA in the large intestine of biopsy specimens from patients with ulcerative colitis and Crohns disease. It appeared that the presence of at least two herpes viruses were needed to cause pathology.

(iv). Maliqnancy

A study by Ruther (Tumordiagnostik & Therpaie 1994 Vol. 15 No 14, pp 121-127) examined biopsy specimens from patients with bronchial carcinoma, pleural mesothelioma, sarcoid and asthma. He found a significant proportion were infected with several herpes viruses (HSV 1 and 2, HHV 6 and EBV) compared to no detectable virus in healthy controls. He suggested that because viruses of the herpes group can play a part in malignant transformation of cells, the role of these viruses as causative agents of malignant diseases in man should be investigated further.

(v). Lymphoproliferative Disorders

It has been known for many years that the herpes virus Epstein Barr is the causative agent in some forms of lymphoma. Further work examining a rapidly fatal lymphoproliferative disorder I bone marrow transplant recipient (Brion et al Nouvelle revue Francaise hematologie 1995 37 6 pp 289-296) showed that herpes virus genome was present in tumoral cells of all analysed specimens including herpes simlex type 1.

All of the above suggest that the role of Herpes simplex virus is not only extremely widespread within the general population but its pathological effects can be just as diverse highlighting the urgent need for effective treatments. It is considered that this invention will provide such effective treatments.

Floridoside is also cytotoxic. Viruses are obligate intracellular parasites and totally dependent upon the macromolecular synthetic processes of the infected host cell for their own reproduction and survival. Viruses are indirectly susceptible, therefore, to the effects of cytotoxic drugs.

The antiviral effect of floridiside may be a consequence of both a direct effect on a particular virus-specific reproductive event(s) and other, virus-non-specific suppressive effects (cytotoxicity).

Viruses are associated with and are known to cause various human cancers. For example:

human papilloma virus and carcinoma of the uterine cervix

human herpes virus 8 and Kaposi's scarcoma

hepatitis C virus and hepatic carcinoma

Epstein Barr virus and Burkitt's lymphoma.

The combined cytologically suppressive (‘cytotoxic’) effects and the antiviral effects of floridoside influence the development and behaviour of cancerous cells. Floridoside suppresses the synthesis of virus specific and host cell proteins.

In order that the invention may be well understood it will now be illustrated with reference to the following example:

### EXAMPLE

Fresh palmaria palmata was first gathered from its salt water environment. The algae was then washed in clean tap water or distilled water and dried at 85° C. until its moisture content was 10% by weight of the algae. The dried algae was preserved for later use by storing at −20° C. until required.

Prior to use, the algae was comminuted by any suitable device (e.g. a blender or pestle and mortar) to produce a fine powder. The powder was then dissolved in water to form the active ingredient, which was incorporated in a pharmaceutical preparation.

1. Use of a floridoside in the treatment of viral infections and neoplastic disease.

2. Use according to claim 1, wherein the floridoside comprises floridoside itself, a water soluble or water miscible salt or ester of floridoside, or an isomeric form of floridoside.

3. A pharmaceutical composition useful in the treatment of viral infections and neoplastic disease comprising an effective amount of a floridoside.

4. A composition according to claim 3, wherein the floridoside comprises floridoside itself or a water soluble or water miscible salt or ester of floridoside, or an isomeric form of floridoside.

5. A composition according to claim 3 or 4, adapted for administration orally, intravenously, or topically.

6. A composition according to claim 5, adapted for administration by oral ingestion.

7. A composition according to any of claims 3 to 6, adapted for administration in the treatment of viruses of the Herpes family.

8. A method of preparing floridoside by extraction from palmaria palmata, the method comprising drying the palmaria palmata to a predetermined water content, comminuting the material to a fine powder and dissolving the powder in water.

9. A method according to claim 8, including the step of storing the powder at or below room temperature.

10. A method according to claim 8 or 9, including the step of centrifuging the solution and recovering the supernatant.

11. A method according to claim 8, 9, or 10, wherein the dried material is mixed with water in the ratio of 50 g:300 ml.

12. A method of treating Herpes virus infections and neoplastic disease in human or animal subjects, comprising applying to the subject a pharmaceutical composition of a floridoside.

13. A method according to claim 12, wherein the application is ingestion.
